(19) United States
(12) Patent Application Publication
(10) Pub. No.: US 2024/0156668 A1
(43) Pub. Date: May 16, 2024

(54) Compression suit for use in the treatment of neurological or psychiatric disorders

(71) Applicant: Neures Polska Sp. z o.o., Warszawa (PL)

(72) Inventor: Leszek Abakumow, Warszawa (PL)

(21) Appl. No.: 18/550,689

(22) PCT Filed: Feb. 14, 2022
(86) PCT No.: PCT/IB2022/051289

§ 371 Date: Sep. 15, 2023

(30) Foreign Application Priority Data
Mar. 16, 2021 (PL) PL437315

(51) Int. Cl. A61H 9/00; A61M 21/00

(52) U.S. Cl. CPC A61H 9/0078; A61M 21/00; A61H 2201/0107; A61H 2201/1238; A61H 2201/1409; A61H 2201/165; A61H 2205/06; A61H 2205/081; A61H 2205/083; A61H 2205/10; A61M 2021/0022

(57) ABSTRACT

The subject of the application is a compression suit (1) for use in the treatment of neurological or psychiatric disorders by exerting pressure on soft tissues, containing a fabric suit (2) for application to the patient's body. The suit is characterised in that it further comprises the pneumatic system for generating and transferring pressure to the soft tissues of the patient, made up of pneumatic inserts (3) for fixing in the areas where the suit interacts with the soft tissues and a pneumatic pump (7) for pumping air into the pneumatic inserts (3) and for regulating the pressure in the pneumatic system. Furthermore, the pneumatic system comprises tubes (5) for supplying air from the pneumatic pump to the pneumatic inserts, whereby the pneumatic inserts are arranged to allow the full range of motion for all movements of limbs and joints. Furthermore, the subject of the application is the use of the compression suit in the treatment of neurological or psychiatric disorders.

The subject of the invention is a compression suit for use in the treatment of neurological or psychiatric disorders and the use of a compression suit.

The solution belongs to the field of devices used in support of treatment of neurological or psychiatric disorders and rehabilitation.

In patent PL219506B1, a rehabilitation swimming suit is disclosed, equipped with a strap to be placed around the front part of the trunk and the back part of the trunk, and longitudinal straps placed with the belt around the trunk. The straps have floating chambers at the height of the cervical, lumbar, thoracic, and sacral spine. Each chamber is equipped with means for filling with gas and means for regulating the volume of gas filling the chamber. The rehabilitation swimming suit disclosed in the patent makes the patient stay afloat on the water surface permanently and stably. Furthermore, the suit allows the rehabilitation of various types of musculoskeletal disorders.

Document PL407901A1 discloses a suit made of a flexible, compressible, breathable material that covers the user's entire body or particular parts of it. The suit is equipped with a flexible system of adjustable lashing straps attached to the surface of the suit and electrostimulation in the form of electrodes and tubes in the structure of the suit material allowing the use of external devices reading or sending electrical impulses from relevant parts of the patient's body. The tension force of the adjustable lashing straps is suitable for inducing straightening, bending, and rotational forces in specific parts of the body, allowing free correction in limbs, trunk, and head mobility.

The present invention is intended to provide a compression suit for use in therapy that activates physiological mechanisms by pressure on the patient's tissues, while ensuring full mobility of the limbs and joints.

The essence of the invention is a compression suit for use in the treatment of neurological or psychiatric disorders by exerting pressure on soft tissues, containing a fabric suit for application to the patient's body.

The suit according to the invention is characterised in that it further comprises the pneumatic system for generating and transferring pressure to the soft tissues of the patient, made up of pneumatic inserts for fixing in the areas where the suit interacts with the soft tissues and a pneumatic pump for pumping air into the pneumatic inserts (3) and for regulating the pressure in the pneumatic system. Furthermore, the pneumatic system comprises tubes for supplying air from the pneumatic pump to the pneumatic inserts, whereby the pneumatic inserts are arranged to allow the full range of motion for all movements of limbs and joints.

Preferably, the suit, according to the invention, further comprises a tubular system to be placed on the back of the patient for transferring pressure to the soft tissues of the patient.

Preferably, in the suit, according to the invention, the tubular system takes the form of two rubber tubes connected to the fabric suit with non-stretchable bands.

Preferably, the suit, according to the invention, comprises pneumatic inserts for fixing on the patient's arms, forearms, thighs, and shanks.

Preferably, in the suit, according to the invention, the fabric suit takes the form of an outer covering for the trunk and limbs of the patient.

Preferably, in the suit, according to the invention, the outer covering of the patient's limbs takes the form of detachable sleeves and legs.

Preferably, in the suit, according to the invention, the pneumatic inserts take the form of air bags placed in pockets sewn onto the fabric suit.

Preferably, the suit, according to the invention, further comprises an abdominal compressor connected to the pneumatic system for exerting pressure on the lower abdomen and the front part of the pelvis.

Preferably, in the suit, according to the invention, the fabric suit comprises an adjusting mechanism in the form of laces, preferably polypropylene ones, for adjusting the compression suit to the patient's body.

Preferably, in the suit, according to the invention, different pressures are present in the pneumatic system.

Preferably, in the suit, according to the invention, pressures in the abdominal compressor, the tubular system on the back, and the pneumatic inserts are different.

Preferably, in the suit, according to the invention, pressures in the abdominal compressor, the tubular system on the back, and the pneumatic inserts are regulated independently of one another.

Preferably, in the suit, according to the invention, pressures in the abdominal compressor, the tubular system on the back, and the pneumatic inserts are regulated dependently on one another.

Furthermore, the essence of the invention is the use of the compression suit in the treatment of neurological disorders.

Preferably, the suit, according to the invention, is used in the treatment of disorders, such as ischemic strokes, Parkinson's disease, cerebral palsy, spinal cord injury, multiple sclerosis, amyotrophic lateral sclerosis, cerebellum post-tumour condition, spinal cord post-tumour condition, peroneal nerve injury, cerebellar ataxia as well as balance and coordination disorders.

Furthermore, the essence of the invention is the use of the compression suit in the treatment of psychiatric disorders.

Preferably, the suit, according to the invention, is used in the treatment of disorders, such as early childhood autism, autism, Asperger syndrome, schizophrenia, senile dementia, and depression.

The construction of the suit according to the invention allows the full range of motion for all movements of limbs and joints, and thus the suit can be used in support of any rehabilitation method.

The advantageous embodiment of the subject of the invention has been shown in greater detail in the following figures whereby:

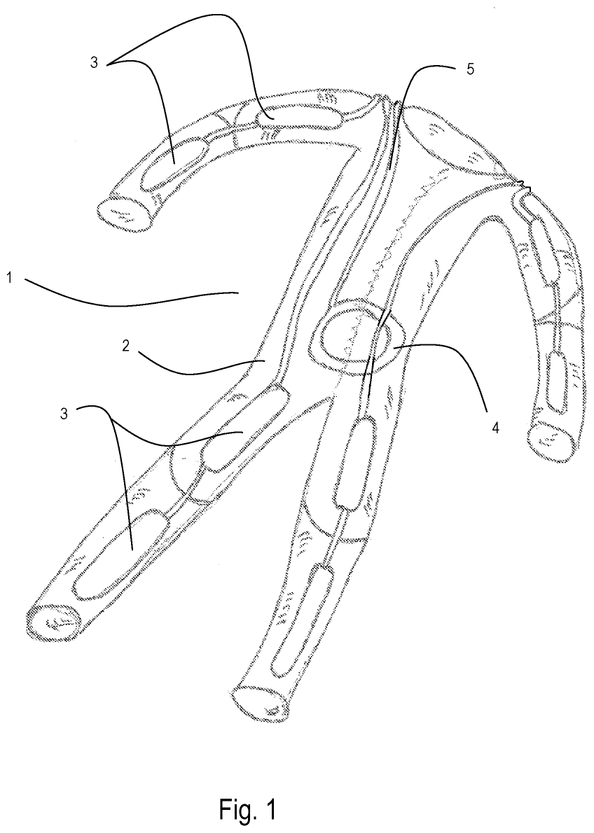

FIG. 1 shows a front view of the compensation suit.

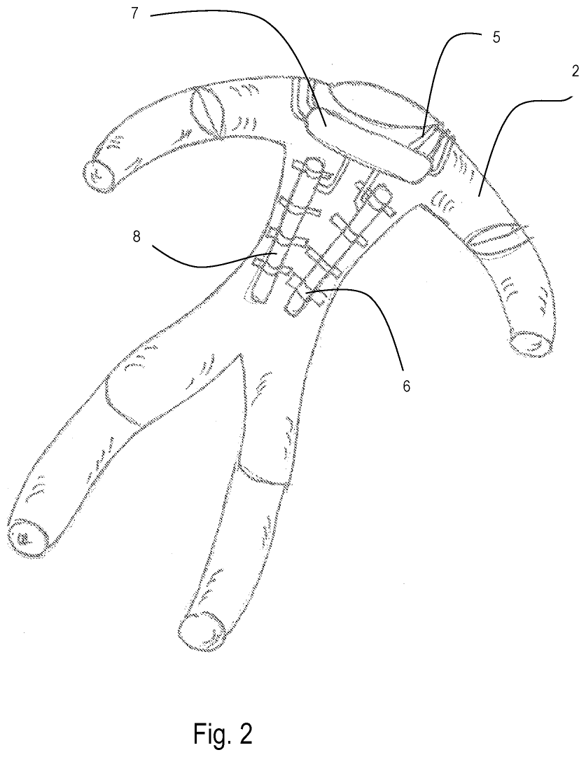

FIG. 2 shows a rear view of the compensation suit.

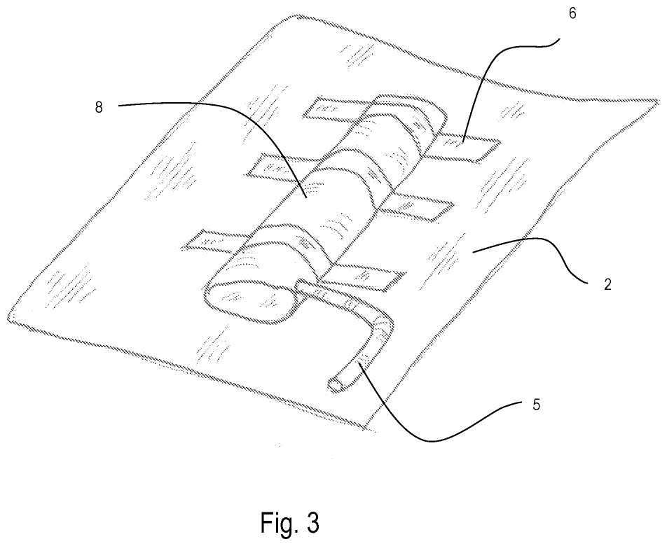

FIG. 3 shows the attachment of the tubular system to the surface of the suit according to the invention.

FIG. 1 shows a front view of the compression suit 1 as according to the invention. The compression suit 1, according to the invention, comprises a fabric part 2 applied to the patient's body, e.g. to the limbs, torso, and head, leaving the feet and hands uncovered. The compression suit 1 further comprises the pneumatic system for generating and transferring pressure to the soft tissues of the patient, made up of pneumatic inserts 3 for fixing at the soft tissue areas and a pneumatic pump 7 for pumping air into the pneumatic inserts 3 and for regulating the pressure in the pneumatic system, and pressure hoses 5. As shown in FIG. 1, air is supplied from the pneumatic pump 7 to the pneumatic inserts 3 via the hoses 5, whereby the pneumatic inserts 3 may be connected in series to form a pneumatic system in star configuration.

In star configuration of the pneumatic system, the pressure in different arms of the system can be adjusted independently, resulting in different pressures in the pneumatic system. Pressures in the abdominal compressor, the tubular system on the back, and the pneumatic inserts do not need to be the same; preferably, they are different. Preferably, pressures in the abdominal compressor, the tubular system on the back, and the pneumatic inserts are regulated independently of one another or based on a dependence determined by the adopted use of the suit. The construction of the suit increases the therapeutic effects by exerting different pressures on soft tissues located in different areas of the body of the person using the suit.

The pneumatic inserts 3 are arranged to allow the full range of motion for all movements of limbs and joints, i.e. they are placed in areas other than the joints of the person wearing the compression suit. The increase in pressure in the pneumatic system exerts pressure on the soft tissues of the exercising person and at the same time does not block the mobility of the joints so that the person wearing the compression suit can freely perform therapeutic exercises. For example, the pneumatic inserts 3 may be attached to the patient's arms, forearms, thighs, and shanks. The pneumatic inserts 3 may take the form of air bags placed in pockets sewn onto the fabric suit. When the pneumatic pump 7 is started, air is pumped into the pneumatic inserts through the air supply lines or hoses 5. The pressure level can be selected using the control panel. It is also possible to select different pressure values depending on the part of the body on which pressure is exerted. When the set pressure is reached, the pump 7 switches to the state of monitoring the pressure in the system and keeps the pressure at a constant level by adding air when a drop in pressure is detected. After filling with air, the air pads 3 begin to increase in size and the fabric of the fabric suit 2 begins to press more firmly against the patient's body, subjecting the soft tissues to precisely adjusted pressure. This pressure causes body reactions that stimulate regenerative and compensatory processes in the nervous system, especially in the brain. As shown in FIG. 1, the pneumatic system of the suit may be further equipped with an abdominal compressor 4 connected to the pneumatic system for exerting pressure on the lower abdomen and the front part of the pelvis.

FIG. 2 shows a rear view of the compensation suit. Preferably, the compression suit 1 may be equipped with an additional tubular system 8 placed on the back of the patient for transferring pressure to the soft tissues of the patient. The tubular system 8 takes the form of two rubber tubes connected to the fabric suit with non-stretchable bands 6. Air is pumped into the rubber tubes by means of the air pump 7 and supplied by means of tubes or hoses 5. Adding air increases the diameter of the rubber tubes, generating forces on the non-stretchable bands that exert additional pressure on the body tissues of the patient. The compression suit may further comprise an adjusting mechanism in the form of polypropylene laces for adjusting the compression suit to the patient's body. The suit may also be equipped with a metal lock.

In one of the embodiments, the fabric suit 2 has detachable sleeves and legs to allow the suit to fit different body parameters, in particular different limb lengths, e.g. a patient with long arms but short legs.

FIG. 3 shows the attachment of the tubular system to the surface of the suit according to the invention. The rubber tubes of the tubular system 8 are attached to the surface of the fabric suit 2 by means of non-stretchable fabric bands 6 using joints to ensure that the tubular system 8 is firmly attached to the fabric suit 2.

The compression suit according to the invention is used in the treatment of neurological and psychiatric disorders. Studies have confirmed the beneficial effect of the compression suit therapy for the following disease entities: The list of disease entities for which the therapeutic effect is beneficial (confirmed by research) includes ischemic strokes, Parkinson's disease, cerebral palsy, spinal cord injury, multiple sclerosis, amyotrophic lateral sclerosis, cerebellum post-tumour condition, spinal cord post-tumour condition, peroneal nerve injury, cerebellar ataxia, balance and coordination disorders.

One of the mechanisms affecting the patient's body in the use of the suits according to the invention is the increased blood supply to cerebral structures (confirmed by research) giving patients with psychological and psychiatric disorders a chance to return to normal. The invention has a beneficial effect for the treatment of the following disease entities: early childhood autism, autism, Asperger syndrome, schizophrenia, senile dementia, and depression.

After filling with air the pneumatic inserts of the suit according to the invention, the fabric of the compression suit begins to press more firmly against the patient's body, exerting pressure on the body tissues. The pressure activates many physiological mechanisms that have a therapeutic effect on the patient with neurological problems. These include the release of neurotransmitters from the adrenal glands, activation of the proprioceptive system, and stimulation of interoreceptors and exteroreceptors. These stimuli cause changes in brain function and improve blood supply to areas of the brain that are ischaemic due to malfunctions in the body's control systems. Furthermore, neurotransmitters cause changes in the activity of neurological processes and stimuli exerted on the receptors normalise brain function.

1. A compression suit (1) for use in the treatment of neurological or psychiatric disorders by exerting pressure on soft tissues, containing a fabric suit (2) to be applied to the patient's body,
characterised in that it further comprises
a pneumatic system for generating pressure and transferring it to the soft tissues of the patient, made up of
pneumatic inserts (3) for fixing in areas where the suit interacts with soft tissues, and
a pneumatic pump (7) for pumping air into the pneumatic inserts (3) and to regulate the pressure in the pneumatic system,
tubes (5) for supplying air from the pneumatic pump to the pneumatic inserts, whereby
the pneumatic inserts are arranged to allow the full range of motion for all movements of limbs and joints.

2. The suit, according to claim 1, characterised in that it further comprises a tubular system (8) to be placed on the back of the patient for transferring pressure to the soft tissues of the patient.

3. The suit, according to claim 2, characterised in that the tubular system takes the form of at least two rubber tubes (8) connected to the fabric suit with non-stretchable bands (6).

4. The suit, according to claim 1, characterised in that it comprises pneumatic inserts (3) for fixing on the patient's arms, forearms, thighs, and shanks.

5. The suit, according to claim 1, characterised in that the fabric suit (2) takes the form of an outer covering for the trunk and limbs of the patient.

6. The suit, according to claim 5, characterised in that the outer covering of the patient's limbs takes the form of detachable sleeves and legs.

7. The suit, according to claim 1, characterised in that the pneumatic inserts (3) take the form of air bags placed in pockets sewn onto the fabric suit (2).

8. The suit, according to claim 1, characterised in that it further comprises an abdominal compressor (4) connected to the pneumatic system for exerting pressure on the lower abdomen and the front part of the pelvis.

9. The suit, according to claim 1, characterised in that the fabric suit (2) comprises an adjusting mechanism in the form of laces, in particular polypropylene ones, for adjusting the compression suit to the patient's body.

10. The suit, according to claim 1, characterised in that different pressures are present in the pneumatic system.

11. The suit, according to claim 10, characterised in that pressures in the abdominal compressor, the tubular system on the back, and the pneumatic inserts are different.

12. The suit, according to claim 1, characterised in that pressures in the abdominal compressor, the tubular system on the back, and the pneumatic inserts are regulated independently of one another.

13. The suit, according to claim 1, characterised in that pressures in the abdominal compressor, the tubular system on the back, and the pneumatic inserts are regulated dependently on one another.

14. The compression suit, as in claim 1, for use in the treatment of neurological disorders.

15. The compression suit, according to claim 14, for use in the treatment of disorders, such as ischemic strokes, Parkinson's disease, cerebral palsy, spinal cord injury, multiple sclerosis, amyotrophic lateral sclerosis, cerebellum post-tumour condition, spinal cord post-tumour condition, peroneal nerve injury, cerebellar ataxia as well as balance and coordination disorders.

16. The suit, as in claim 1, for use in the treatment of psychiatric disorders.

17. The suit, according to claim 16, for use in the treatment of disorders, such as early childhood autism, autism, Asperger syndrome, schizophrenia, senile dementia, and depression.
